(19) United States
(12) Patent Application Publication
(10) Pub. No.: US 2023/0207079 A1
(43) Pub. Date: Jun. 29, 2023

(54) SMARTPHONE-ENABLED SYSTEMS, METHODS, DEVICES, AND COMPUTER PROGRAM PRODUCTS FOR COLLECTING PERFORMANCE DATA AND USING A SELFIE TO GENERATE SELF-VERIFYING DATA SETS

(71) Applicant: CheckApp Inc. (a Canadian federal corporation), Ottawa (CA)

(72) Inventor: Robert Bender, Ottawa (CA)

(73) Assignee: CheckApp Inc. (a Canadian federal corporation), Ottawa (CA)

(21) Appl. No.: 18/088,240

(22) Filed: Dec. 23, 2022

Related U.S. Application Data
(60) Provisional application No. 63/293,546, filed on Dec. 23, 2021.

(51) Int. Cl. G16H 10/60; G06F 21/32; G16H 10/20; G06F 21/60; G16H 50/30

(52) U.S. Cl. CPC G16H 10/60; G06F 21/32; G16H 10/20; G06F 21/602; G16H 50/30

(57) ABSTRACT

Disclosed embodiments relate to a method for creating unique data sets which include and integrate subsets allowing validation of origin (subject), measurement of relevant performance attributes and confirmation of compliance. In one embodiment, a method may be performed by a computing device, CD, for creating data sets. The method may include opening a testing module stored within the CD. The method may further include loading a series of instructions from the testing module. The method may further include creating a first data set based on the instruction. The first data set may include a first biometric sample of the user corresponding to a biometric identifier of the user, a first time stamp marking the time of the creation of the first data set, an internet protocol (IP) address of the CD, and an one or more first test data points requested by the instructions.

### CROSS-REFERENCE TO RELATED APPLICATIONS

This application is a non-provisional of, and claims the priority benefit of, U.S. Prov. Pat. App. No. 63/293,546 filed Dec. 23, 2021, which is hereby incorporated by reference in its entirety.

### TECHNICAL FIELD

Aspects of the present disclosure relate to smartphone-enabled systems, methods, devices, and computer program products for collecting performance data and using a selfie to generate self-verifying data sets.

### BACKGROUND

Data collection plays a critical role in assessing the health outcomes of individuals. A general problem in collecting and evaluating individual performance data is that there is a weak linkage between the data, the individual subject and time (of stimulus presentation and or data collection). This results in unreliable data and consequently a need for error detection, data correction and significant errors in the primary data sets. Consequences range from outright loss of data to the need to increase the sample size. These problems are well known in medical and consumer product testing and are typically addressed by having third-party monitors confirm data collection and ‘dosing’ or use protocols. However, third-party monitors and compliance checking form a significant cost element in any test or monitoring program. Further, monitors themselves can be a source of errors in recording data.

Traditionally recruitment and data collection has been done with qualified monitors (often RNs) who are tasked with data collection and reporting. Errors at any step of the process impact the quality of the data sets. The costs associated with the use of monitors make dense sampling impractical which in turn increases the need for lengthy trials in many cases where progression signals are noisy. Trials/tests involving multiple sites and outpatients or healthy volunteers have significant logistics. Further, trials designed to establish performance in specific attributes require that sample size and measurement errors be quantified. Commonly errors are introduced by separating data recording from test performance by keeping event logs which are updated asynchronously. Failure of subjects to comply with test schedules is often masked by post facto amendment of the individual event logs.

### SUMMARY

A method for conducting trials or tests which unites data collection with simultaneous verification of source and compliance is provided. It is general in its utility since it can be adapted to multiple data collection modalities. The systems, methods, devices, and computer program products described herein provide for the simultaneous utilization of the multiple sensing and display capabilities of the current generation of smart phones in order to create unique data sets which include and integrate subsets allowing validation of origin (subject), measurement of relevant performance attributes and confirmation of compliance.

In one embodiment, a method performed by a computing device (CD) for creating data sets is disclosed. The method may include opening a testing module stored within the CD. The method may further include loading a series of instructions from the testing module. The method may further include creating a first data set based on the instructions, wherein the first data set includes a first biometric sample of the user corresponding to a biometric identifier of the user, a first time stamp marking the time of the creation of the first data set, an internet protocol (IP) address of the CD, and an one or more first test data points requested by the instructions.

In another embodiment, an enrolled subject (consented and validated as biometric (selfie)) with a smart phone is given one or more apps which may run a trial module. The trial module may create a data set which includes a highly reliable biometric and time stamp. The trial module may further include self-initiated or push-notification driven measurements to be made within the structure of the trial protocol. Data sets originating from the device (per IP address) may be verified by the embedded biometric as well as the time stamp and content.

An advantage of the embodiments disclosed herein include the combination of compliance, subject identity and dosing verification in a single self-administered interaction which is uniquely achieved through the linkage of data provided by the data collection device. This same linkage allows administration of test instruments and validation of data collection (source ID and integrity) to be mediated by smart phone apps which integrate compliance validation and data acquisition. Further, there are advantages of linked or self-verifying data sets in the setting of clinical or consumer product testing relative to conventional methodologies which rely on external validation by ‘monitors’ who are in turn a significant source of errors in data collection.

In particular, the elimination of ‘live’ monitors at data collection sites can lower the costs of trials which depend on multiple data collection events by more than two logs. This is an enabling change in relation to economic feasibility of such trials.

### BRIEF DESCRIPTION OF THE DRAWINGS

The accompanying drawings, which are incorporated herein and form part of the specification, illustrate various embodiments.

FIG. 1 illustrates a system according to an embodiment for data collection.

FIG. 2 illustrates an exemplary data set.

FIG. 3 is a flow chart illustrating a process, according to an embodiment, for creating data sets.

FIG. 4 is a flow chart illustrating a process, according to an embodiment, for creating data sets.

FIG. 5 is a block diagram of a computing device, according to some embodiments.

### DETAILED DESCRIPTION

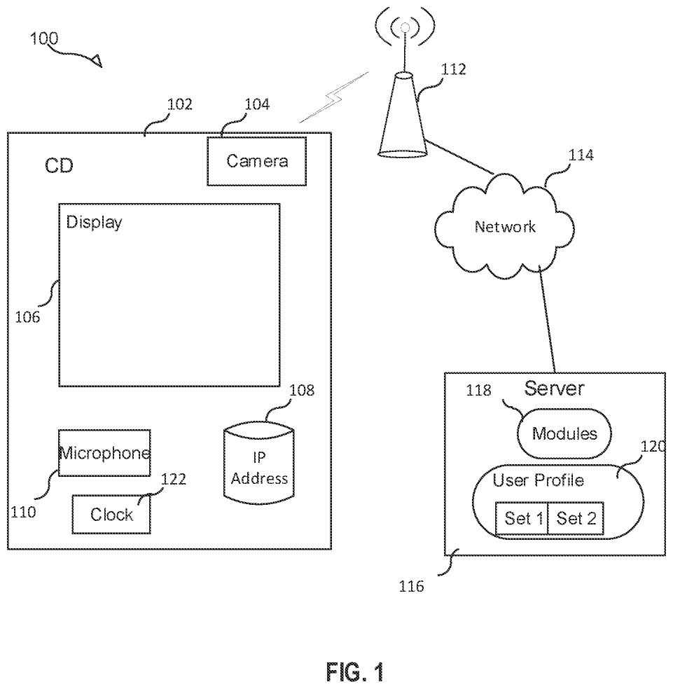

FIG. 1 illustrates a system (100) according to an embodiment for data collection. The system 100 may include a computing device (CD) 102. The CD 102 may be a smart phone, IPad, personal computer, or other electronic device that has internet connectivity and is capable of running applications. The CD 102 may further include a display 106. In some embodiments, the display 106 may include a touch screen. In other embodiments, the CD 102 may include a key board, voice-to-text capabilities, or other means routinely used in the art to allow a user to interact with a computing device.

The CD 102 may also include a microphone 110 and a storage unit 108. The storage unit 108 may have stored within it, an IP address unique to the CD 102. The CD 102 may also include a clock 122.

The CD 102 may further include a multitude of sensors. For example, the CD 102 may include a camera 104 and a microphone 110. In some embodiments, the camera 104 may include multiple cameras such as a front and rear-facing camera. In further embodiments, the camera 104 may include video recording capabilities.

The CD 102 may also have internet connection capabilities and be able to connect to a network 114. In some embodiments, the CD 102 may connect to the network 114 via a base station 112. In other embodiments, the CD 102 may connect to the network 114 via a Wi-Fi connection. Once connected to the network 114, the CD 102 may download an application from a server 116 within the network 114. In other embodiments, the application may be downloaded from a third party server such as the Google App store. The server 116 may further include one or more trial modules 118. Each trial module 118 within the server 116 may correspond to a different trial that can be run by the application. The CD 102 may downloaded a particular trial module 118 from the trial modules 118 that is specific to the trial the user is participating within. In some embodiments, the trial module 118 that is downloaded may be selected by a specific key code being entered into the application.

The server 116 may further include a user profile 120. The user profile 120 may be unique to a specific user of the CD 102. The user profile 120 may be linked to the user of the CD 102 in a number of ways. In one embodiment, the user may connect to the server 116 and transport his IP address to the server 116. The server 116 may then link the IP address to the user profile 120. In other embodiments, the user profile 120 may further include a biometric identifier of the user. Any variation of a biometric identifier such as images of the user, voice recordings of the user, images of the user's hand, or fingers prints of the user may be linked to the user profile 120. To create the biometric identifier, the user may be required to generate a biometric sample from the CD 102 and transfer the biometric sample to the server 116. That is the biometric identifier may represent a broad class such as images of the user, and the biometric sample may represent a particular image of the user.

The trial module 118, once downloaded to the CD 102, may include instructions for the user on how to conduct the trial including creating necessary data sets for the trial. In some embodiments, the trial module 118 may further include a tutorial to train the user on how to conduct the trials including creating data sets.

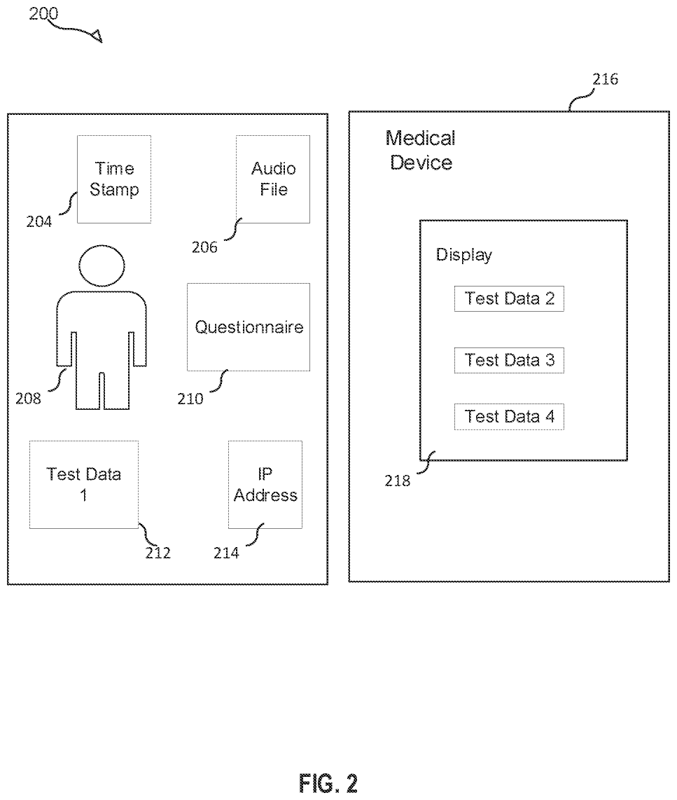

The user may use any of the sensors within the CD 102 including the camera 106 and microphone 110 when creating data sets. The created data sets may include a biometric of the user, a time stamp marking the time of the creation of the data set, an internet protocol (IP) address of the CD, and one or more test data points requested by the instructions. An exemplary discussion of a data set 200 is provided in FIG. 2.

After creating the data set, the application may check the data set for completeness. Checking the data set for completeness may involve determining whether the biometric sample attached to the data set meets a quality threshold. It may further involve determining whether the requested data points were provided.

After determining that the data set is complete, the CD 102 may send the data set to the server 116. In some embodiments, the data set may be encrypted before being transferred to the server 116.

Once the server 116 receives the data set from the CD 102, the server 116 may decrypt the data set if said set was encrypted. The server 116 may then perform verification to determine whether the received data set should be linked to the user profile 120. Verification may involve determining whether the IP address attached within the received data set matches an IP address assigned to the user profile 120. The verification process may further involve determining whether the biometric sample provide in the receive data set matches a biometric identifier assigned to the user profile 120. Once verified, the received data set may be matched with other data sets already attached to the user profile 120.

For example, within the user profile 120, Set 1 may represent a first data set that was received from the CD 102. A second data, Set 2 may have also been received the CD 102. Set 1 and Set 2 may be linked into one data set within the user profile 120. The order in which Set 1 and Set 2 are linked may based on the time of receipt of the data sets or the time stamps included within the data sets. The combination of Set 1 and Set 2 may represent a report of the trial results for the user attached to the user profile 120.

FIG. 2 illustrates an exemplary data set 200.

When creating the data set 200, a user of the CD 102 may first load trial instructions that may have accompanied the trial module 118 downloaded onto an application running on the CD 102.

The created data set 200 may be self-verifying by including a biometric. A self-verifying data set is any data set that contains one or more functions which contain the correct answer to function or feature that can be tested. Typically, data sets comprise multiple independent functions and thus require external data for validation. By linking data collection modalities, data sets can be made self-verifying. An advantage of self-verifying data sets is that self-verifying data sets do not require external (secondary) validation of data collection and that confirmation is internal and not based on the user interface or user action.

Further, using self-verifying data sets can improve both data reliability and lower costs. For example, clinical trials require the creation of detailed data logs that cover a user of a drug and corresponding symptoms. By uniting reported data with a secure biometric, a data set which can verify protocol compliance and eliminate recording or attribution errors may be created.

In some embodiments, the data set 200 may include a biometric sample that corresponds to a biometric identifier of the user. For example, the biometric identifier of the user may be an image 208 of the user. Accordingly, when creating the data set 200, the user may take a selfie and attach this particular image 208 as a biometric sample of the user.

In other embodiments, the biometric identifier for the user may be a voice print of the user. Accordingly, the user may record their voice with the microphone 110 in order to create an audio file 206 that may serve as a biometric sample within the data set 200.

Test data 1 212 may represent one or more data points that the user is meant to record in accordance the particular trial module 118 that is being used. Instructions loaded from the trial module may be provided to the detail which test data points are required. In some embodiments, the test data 1 212 may be recorded with an image. For example, the trial module 118 may be designed to track the overall pulmonary function status of a user taking prescribed medicine. To create the data set 200, the user may take a selfie 208 (biometric sample) and include within the selfie 208 a serialized dose of the prescribed medication (test data 1) 212 to verify compliance.

Further, while the audio file 206 may be used as biometric, it also may be used to store test data within the data set. Using the previous example, the audio file 206 may be included within the data set 200 along with the serialized dose of the prescribed medication 212. The audio file 206 may comprise a simple acoustic recording of a scripted breathing exercise. Based on the data set 200, doctors can look for any changes in pulmonary function status in reaction to taking the medication, changes of which may be indicative of need for more directed intervention.

The data set 200 may further include an IP address 214. The IP address 214 may be unique to the CD 102. The data set 200 may further include a time stamp 204. The time stamp 204 may be embedded within the data structure and include the time and date at which the data structure was created by using the clock 122.

In some embodiments, the data set may further include a questionnaire 210. The questionnaire 210 may include a series of questions or challenges included within the trial module or trial module instructions that are displayed on the display 106. Further, the clock 122 may be used to record and store the time it takes the user to respond to each question or solve each challenge. Additionally, in some embodiments, the display 106 may include touch screen capabilities. The additional touch screen capabilities may allow for a greater range in the types of questionnaires 206 that can be included while also providing a simple, easy to understand, means for a user to input their response. For example, the testing module 118 may include testing the reaction time of a patient while on a particular medicine. The questionnaire 210 may further include displaying objects on the display 106 and having the user select the proper object by using the touch screen capabilities of the display 106. The time it takes the user to correctly select the right object may be recorded by the clock 122 and included within the data set 200.

In some embodiments, based on the instructions provided, medical devices 216 may be used to generate health measurements. If the health device 216 is able to connect with the CD 102 such as through (but not limited to) Bluetooth, then the data may be stored within the data set 200. In other embodiments, the health measurement devices 216 may be unable to connect to the CD 102. In such instances, images of the display 218 of the health measurement device 216 may be taken with the camera 104 and included within the data set 200.

The display 218 may show relevant health measurements test data 2, test data 3, and test data 4. By including the image of the display 218 of the medical device 216 into the data set 200, test data 2, test data 3, and test data 4 of the medical device 216 can be captured. For example, blood pressure measurement devices generally have a visual display which can be photographed with a smart phone for data capture.

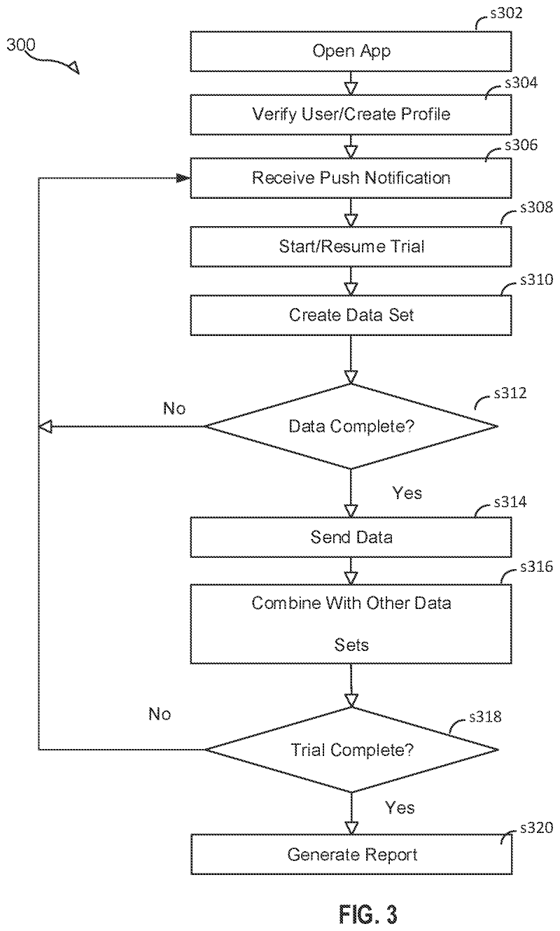

FIG. 3 is a flow chart illustrating a process 300, according to an embodiment, for creating data sets.

Prior to step s302, a user profile may be created. In some embodiments, the user profile may be created by an administrator. In further embodiments, the user profile may be linked to a biometric identifier already obtained from a biometric identifier of the user. For example, a user may apply to participate within a clinical trial. The administrator may then create a user profile for each participant within the clinical trial. Further, facial recognition technology may be applied to a photo of the user (biometric sample) to attach a biometric identifier to the user's profile. In further embodiments, the user may be provided a key code. The key code may be uniquely linked to the user's created profile. In further embodiments, the key code may be used to download the application.

At step s302, the user may open the application. In some embodiments, the user may enter a key code into the application. The entered key code may identify a particular trial module to be loaded into the application. In other embodiments, the user may individually select within the application a trial module to be loaded into the application. In some embodiments, the trial module may be downloaded from a server.

At step s304, the user may be asked to create a user profile if it has not already been created. Creating the user profile may involve the user providing a biometric sample such as selfie of the user in order to create a biometric identifier attached to the user profile. Further, the IP address of the computing device used may also be attached to the user profile. If the user profile has already created, the user may be asked to verify that it has access to the user profile. The verification process may involve the user entering a secure key code or providing a biometric sample such as picture of the user if a biometric identifier has already been applied to the user profile. After verification, the IP address of the computing device used by the user may be attached to user profile.

At step s306, the CD may receive a push notification. Push notifications may be used to notify or remind the user when to create data sets. For example, the user may be participating in a drug clinic in which the user needs to take medicine regularly at three times a week and record the taking of the medicine within data sets. Push notifications may be sent to the user's computing device three times a week to remind the user to take their medication and create the relevant data sets.

At step s308, the user may start the trial within the application. When starting the trial, the user may receive a series of instructions detailing how to create the data sets for the purpose of the trial. In some embodiments, the instructions may further include a tutorial to teach the user how to create the data sets. Non-limiting examples of trials that can be undertaken include clinical trials, and chronic disease monitoring health monitoring of individuals.

For clinical trials, within conventional practice, daily log sheets are filled in to provide records of compliance with the experimental protocol and actual consumption. Monitors typically check logs against consumption (empty container count) on a frequent (weekly) basis and performance testing is done on a sparse sampling basis by monitors. By serializing (blinded) unit doses and taking a ‘selfie’ in which the dose serial number and participant are captured with a time stamp and IP address a self-verifying data set is created and can be incorporated (merged with) similarly self-verifying data sets of test results in a time series. The resultant data set provides a reliable record of compliance free of inadvertent errors (post factor log data and omissions). Further, since compliance can be achieved in real time of the trial, any deviations from protocol can be addressed (and remedied) within a time frame which avoids loss of a participant.

For disease monitoring, diseases such as asthma, COPD or cognitive disorders or congestive heart failure are characterized by slow progression and often punctuated by acute episodes of distress. Diagnosis and assessment of chronic diseases often requires sophisticated measurements but once a condition is established there is a need to track progression in order to adjust treatment programs or avoid sudden exacerbations that cause a disruptive and costly need for emergency care. Failure of medication compliance is a common cause of deviation from a planned trajectory for many patients. Push notifications can help ensure that a patient follows proper medication compliance, while the resulting data sets may provide an accurate record of whether the patient is keeping medication compliance.

At step s310 the user may create a data set. The data set may include biometric sample of the user. The biometric sample may allow the data set to be self-verifying. Examples, of biometric samples that may be included within the data set include selfies of the user, images of the users hand, fingerprints of the user, and a voiceprint of the user.

The data set may further include test data points based on the type of trial. Types of test data points may be generated by a computing device such a smart phone and can include but are not limited images showing administration of serialized test products, health measurements that may be entered into the application running the test module or included within an image, audio recordings that cover the users breathing of speech patterns, and users responses to questionnaires. In further embodiments, health measurement devices other than the computing device running the application may be used. In some embodiments, the health devices may be able to connect to the communication device, and the health measurements by those devices may be added into the structured data sets. In other embodiments, the health devices may be unable to connect to the health measurement devices. An image of the display of the health devices showing the health measurements may be taken included within the data sets.

At step s312, application may check the created data set for completeness. In some embodiments, determining whether the data set is complete involves determining whether the biometric sample matches a certain quality threshold. For example, if the data set is an image of a user, the image quality may have to meet certain image quality requirements. In other embodiments, such as when the biometric is a voice print, the voice print may have to meet certain sound quality requirements. In some embodiments, verifying the data set may further include determining whether all relevant data measurements have been entered. For example, if the user was supposed to enter heart rate measurements, the application will check to ensure those measurements have been entered.

If the data set is not complete, the process may return to step s306. That is the application may receive a push notification notifying them that the data set is incomplete. In some embodiments, the push notification may inform the user which portion of the data set was incomplete such as the biometric sample being insufficient. The user may then have to repeat steps s308 through s310 in order to correct the incomplete data structure.

At step s314, the data set is transmitted to the server containing the user profile. The data set may be transmitted to the server via an internet connection. Prior to transmission, the data set may be encrypted. The server may then decrypt the received data set.

After decrypting the data set, the server may perform verification of the data set. In performing verification, the server may determine whether the biometric sample and IP address stored within the data set align with the biometric identifier and IP address attached to the user profile.

At step s316, the server, after determining that the data set is associated with the user profile, may attach the data set to the any other data sets already attached to the user profile. The order in which the data sets are attached may be based on the order in which the server receives the data sets. In other embodiments, such as, if multiple data sets are sent at one time to the server, the received data sets may be combined in order based on the time stamp attached to each data set.

At step s318, the trial module being run may determine whether the user has completed the trial. If the user has not completed the trial, the user may receive a push notification and the process may repeat through steps s306 through s316. In some embodiments, a trial may be scheduled to last over a long period of time. Accordingly, the push notification received by the user to continue the trial may be spaced out. For example, the user may be participating in a clinical trial that lasts for eight weeks. The trial may be designed so that the user needs to submit a data set every week. Once the user has submitted a data set, the user may not receive a push notification to submit a new data set until the next week.

At step s320 a report is generated. The report may include all of the linked of the data sets attached to the user profile within the server. In some embodiments, the report generated may not be a final report. That is the report may be an interim report designated by the particular testing module being operated by the application. In such instances, subsequent testing as dictated by the testing module instructions may be performed. In some embodiments, a final report may be generated after providing the interim report. The final report may include all the linked data sets within the user profile including the data sets previously provided in the interim report.

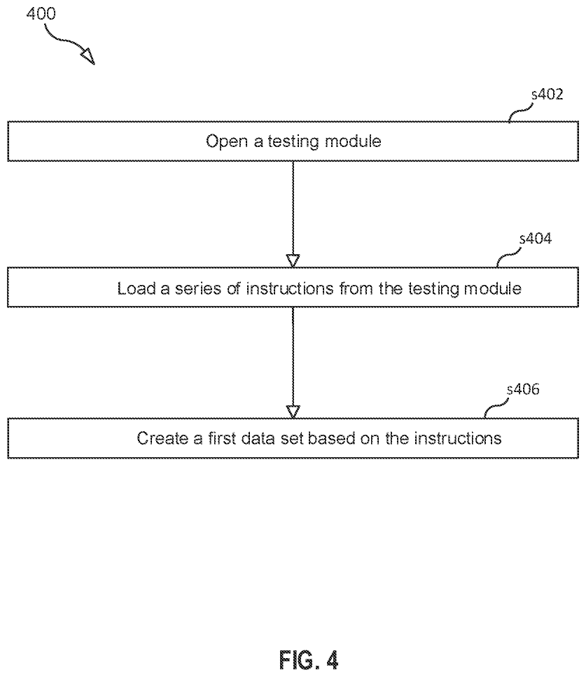

FIG. 4 is a flow chart illustrating a process 400, according to an embodiment, for creating data sets

At step s402, a user may open a testing module within a CD. The testing module may include a series of instructions for the user to follow. In some embodiments, the testing module may be downloaded from a server and run by an application within the CD. The specific trial module downloaded may be determined by a key code. The key code may be entered by the user within the application or scanned through a QR code.

In some embodiments, a user profile may be created within the server. The user profile may identify a specific user. The user profile may be created by an administrator within the server. The user profile may be linked to either the user's IP address or biometric or both.

At step s404, the user may load a series of instructions from the testing module. The instructions may detail how the trial is to be conducted and what steps the user needs to take to create data sets.

In some embodiments, the instructions may include a tutorial that is designed to teach the user how to create the desired data sets and transfer the data sets to the server using the application running the testing module.

At step s406, the user may create a data set based on the instructions loaded from the testing module. The first data set may include a biometric sample of a user corresponding to a biometric identifier of the user. The biometric identifier of the user may be one of an image of the user, a voice print of the user, a finger print of the user, or an image of a hand of the user. The biometric sample may be a particular sample within the type of biometric identifier. For example, if the biometric identifier is an image of the user, the biometric sample may be a particular image of the user. In another example, the biometric identifier may be a voice print of the user. In yet another example, the data set may include an audio file of the user speaking that serves as a biometric sample.

The first data set may further include a first time stamp marking the time of the creation of the first data set, an internet protocol (IP) address of the CD, and one or more first test data points requested by the instructions. In some embodiments, the first test data points may be an image of a serialized test product that confirms its administration. In other embodiments, the test points may include test data specific to a test measurements. For example, a heart rate measurement.

In some embodiments, the user may send an encrypted version of the first data set to the server. The server may be configured to decrypt the encrypted version of the first data set and assign the first data set to the user profile based on at least one of the IP-address and the biometric sample within the first data set.

In some embodiments, the user may a create a second data set that includes a second biometric sample of the user corresponding to the biometric identifier of the user, a second time stamp marking the time of the creation of the second data set, the IP address of the CD, and one or more second test data points. In further embodiments, the user may send an encrypted version of the second data set to the server. The server may be configured to decrypt the second data set and combine the second data set with the first data set assigned to the user profile based on at least one of the IP-address and the biometric sample within the first data set.

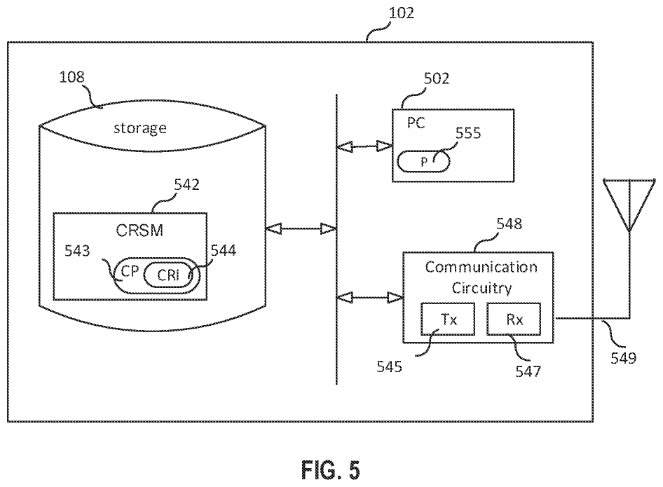

FIG. 5 is a block diagram of computing device (CD) 102, according to some embodiments. As shown in FIG. 5, CD 102 may comprise: processing circuitry (PC) 502, which may include one or more processors (P) 555 (e.g., one or more general purpose microprocessors and/or one or more other processors, such as an application specific integrated circuit (ASIC), field-programmable gate arrays (FPGAs), and the like); communication circuitry 548, which is coupled to an antenna arrangement 549 comprising one or more antennas and which comprises a transmitter (Tx) 545 and a receiver (Rx) 547 for enabling CD 102 to transmit data and receive data (e.g., wirelessly transmit/receive data); and a storage unit (a.k.a., “data storage system”) 108, which may include one or more non-volatile storage devices and/or one or more volatile storage devices. In embodiments where PC 502 includes a programmable processor, a computer readable storage medium (CRSM) 542 may be provided. CRSM 542 may store a computer program (CP) 543 comprising computer readable instructions (CRI) 544. CRSM 542 may be a non-transitory computer readable medium, such as, magnetic media (e.g., a hard disk), optical media, memory devices (e.g., random access memory, flash memory), and the like. In some embodiments, the CRI 544 of computer program 543 is configured such that when executed by PC 502, the CRI causes CD 102 to perform steps described herein (e.g., steps described herein with reference to the flow charts). In other embodiments, CD 102 may be configured to perform steps described herein without the need for code. That is, for example, PC 502 may consist merely of one or more ASICs. Hence, the features of the embodiments described herein may be implemented in hardware and/or software.

While various embodiments are described herein, it should be understood that they have been presented by way of example only, and not limitation. Thus, the breadth and scope of this disclosure should not be limited by any of the above described exemplary embodiments. Moreover, any combination of the above-described elements in all possible variations thereof is encompassed by the disclosure unless otherwise indicated herein or otherwise clearly contradicted by context.

1. A method performed by a computing device, CD, for creating data sets, the method comprising:
opening a testing module stored within the CD;
loading a series of instructions from the testing module; and
creating a first data set based on the instructions, wherein the first data set includes a first biometric sample of the user corresponding to a biometric identifier of the user, a first time stamp marking the time of the creation of the first data set, an internet protocol (IP) address of the CD, and one or more first test data points requested by the instructions.

2. The method of claim 1, the method further comprising:
prior to opening the testing module within the CD, downloading the testing module from a server; and
prior to creating the first data set, creating a user profile that is stored in the server, wherein at least one of the IP address of the CD and the biometric identifier of the user is assigned to the user profile.

3. The method of claim 2, the method further comprising:
sending an encrypted version of the first data set to the server, wherein the server is configured to decrypt the encrypted version of the first data set and assign the first data set to the user profile based on at least one of the IP-address and the biometric sample within the first data set.

4. The method of claim 3, wherein the server is further configured to, after decrypting the data set, verify if the biometric sample within the first data set matches the biometric identifier assigned to the user profile.

5. The method of claim 3, the method further comprising
creating a second data set that includes a second biometric sample of the user corresponding to the biometric identifier of the user, a second time stamp marking the time of the creation of the second data set, the IP address of the CD, and one or more second test data points; and
sending an encrypted version of the second data set to the server, wherein the server is configured to decrypt the second data set and combine the second data set with the first data set assigned to the user profile based at least one of the IP address of the CD and the second biometric sample within the second set.

6. The method of claim 1, wherein the first data set further includes one or more responses to a questionnaire that was included within the testing module.

7. The method of claim 6, wherein each response to the questionnaire further includes a response time that measures the amount of time it took the user generate each response to the questionnaire.

8. The method of claim 1, wherein the first data set further comprises an audio recording generated by the user.

9. The method of claim 1, wherein the biometric identifier of the user is any one of an image of the user, a voice print of the user, a finger print of the user, or an image of a hand of the user.

10. The method of claim 1, wherein the one or more first data points includes an image of a serialized test product.

11. The method of claim 1, wherein the first data set further includes an image of a health measurement device displaying one or more health measurements of the user performed by the health measurement device based on the instructions.

12. A computing device (CD), the CD comprising:
a processor; and
a non-transitory computer readable memory coupled to the processor, wherein the system is configured to:
open a testing module stored within the CD;
load a series of instructions from the testing module; and
create a first data set based on the instructions, wherein the first data set includes a first biometric sample of the user corresponding to a biometric identifier of the user, a first time stamp marking the time of the creation of the first data set, an internet protocol (IP) address of the CD, and one or more first test data points requested by the instructions.

13. The CD of claim 12, wherein the CD is further configured to:
prior to opening the testing module within the CD, download the testing module from a server; and
prior to creating the first data set, create a user profile that is stored in the server, wherein at least one the IP-address of the CD and the biometric identifier of the user is assigned to the user profile.

14. The CD of claim 13, wherein the CD is further configured to:
send an encrypted version of the first data set to the server, wherein the server is configured to decrypt the encrypted version of the first data set and assign the first data set to the user profile based on at least one of the IP address and the biometric sample within the first data set.

15. The CD of claim 14, wherein the CD is further configured to:
create a second data set that includes a second biometric sample of the user corresponding to the biometric identifier of the user, a second time stamp marking the time of the creation of the second data set, the IP-address of the CD, and one or more second test data points; and
send an encrypted version of the second data set to the server, wherein the server is configured to decrypt the second data set and combine the second data set with the first data set assigned to the user profile based at least one of the IP address of the CD and the second biometric sample within the second set.

16. The CD of claim 12, wherein the first data set further includes one or more responses to a questionnaire that was included within the testing module.

17. The CD of claim 12, wherein the one or more first data points includes an image of a serialized test product.

18. The CD of claim 12, wherein the first data set further comprises an audio recording generated by the user.

19. The CD of claim 12, wherein the biometric identifier of the user is any one of an image of the user, a voice print of the user, a finger print of the user, or an image of a hand of the user.

20. A method for creating a linked self-verifying data structure relating to health records, the method comprising:
creating a first data set within a computing device, wherein the first data set comprises a first biometric sample of a user corresponding to a biometric identifier of the user, a first time stamp marking the time of the creation of the first data set, an internet protocol (IP) address of the computing device, and one or more first test data points;
creating a second data set within the computing device, wherein the second data set comprises a second biometric sample of a user corresponding to the biometric identifier of the user, a second time stamp marking the time of the creation of the second data set, the internet protocol (IP) address of the computing device, and one or more second test data points; and
linking the first data set to the second data set based on the first biometric sample within the first data set and the second biometric sample within the second data set both corresponding to the same biometric identifier of the user, wherein linking the first data set to the second data set based on the first biometric sample and the second biometric sample both corresponding to the same biometric identifier creates a verified health record without the use of a monitor.
